(19) United States
(12) Patent Application Publication
(10) Pub. No.: US 2024/0180802 A1
(43) Pub. Date: Jun. 6, 2024

(54) USE OF NEUROSPORENE TO PROTECT THE SKIN FROM THE HARMFUL EFFECTS OF BLUE LIGHT

(71) Applicant: DEINOVE, GRABELS (FR)

(72) Inventors: CORALIE MARTIN, MONTPELLIER (FR); GREGORY BARONIAN, MONTPELLIER (FR); RACHIDA NACHAT-KAPPES, CHATEL-GUYON (FR)

(21) Appl. No.: 17/773,058

(22) PCT Filed: Oct. 30, 2020
(86) PCT No.: PCT/FR2020/051970

§ 371 Date: Apr. 29, 2022

(30) Foreign Application Priority Data
Nov. 2, 2019 (FR) FR1912313

(51) Int. Cl. A61K 8/31; A61K 31/01; A61P 17/02; A61Q 17/04

(52) U.S. Cl. CPC A61K 8/31; A61K 31/01; A61P 17/02; A61Q 17/04; A61K 2800/92

(57) ABSTRACT

The present invention relates to the use of neurosporene to protect the skin from the harmful effects of blue light and/or UVA, to promote epidermal regeneration or renewal, and to stimulate wound healing.

### SUBJECT OF THE INVENTION

The present invention falls within the field of the cosmetic industry and the pharmaceutical industry and relates in particular to the use of neurosporene in new cosmetic or pharmaceutical applications.

### BACKGROUND OF THE INVENTION

Skin ageing is a normal physiological phenomenon which can accelerate under the influence of various intrinsic and extrinsic factors. Intrinsic skin ageing corresponds to the inevitable modifications which affect the skin and are age related. Extrinsic ageing is linked to exterior factors, essentially environmental factors.

The main extrinsic factors are light radiation and in particular ultraviolet radiation. Although for many years UVB radiation was considered to be the only one responsible for damage, the last few years have also demonstrated the harmful effects of UVA radiation.

Just like UVA radiation, it has become apparent that blue light, in particular emitted by LED lamps, computer screens and mobile telephones, causes not only oxidative stress in the skin (Nakashima et al., Free Radic Biol Med. 2017 July; 108:300-310) but also other harmful effects such as keratinocyte toxicity (Liebmann et al., J Invest Dermatol. 2010 January; 130(1):259-69), hyperpigmentation (Regazzetti et al., J Invest Dermatol. 2018 January; 138(1):171-178), epidermal dehydration (Avola et al., Cells. 2018 Nov. 3; 7(11)), impairment of the circadian cycle (Tosini et al., Mol Vis. 2016 Jan. 24; 22:61-72) and mitochondrial functions (Tao et al., Oxid Med Cell Longev. 2019 Aug. 21; 2019:6435364). These effects induce an acceleration of skin ageing and promote in particular the appearance of wrinkles or pigment spots.

The change in behaviors results in an increasing exposure of our skin to blue light. There is therefore an increasing need for cosmetic products capable of countering the harmful effects that this radiation can have on the skin.

### SUMMARY OF THE INVENTION

In order to meet this expectation, the inventors have demonstrated that neurosporene is capable of efficiently countering the various effects associated with blue light and capable of resulting in accelerated skin ageing.

Thus, the present invention relates to the cosmetic use of a composition comprising neurosporene to protect the skin from one or more harmful effects of blue light and/or of UVA radiation, and/or to promote epidermal regeneration or renewal, and/or to stimulate wound healing, in particular to improve the esthetic appearance of a scarred or damaged area of the skin.

Preferably, the composition is a cosmetic composition comprising neurosporene as cosmetic active ingredient.

Preferably, the composition comprises an effective amount of neurosporene, preferably between 0.00001% and 20% by weight of neurosporene relative to the total weight of the composition, more particularly preferably between 0.001% and 10% by weight of neurosporene relative to the total weight of the composition, and preferably more than anything between 0.001% and 2% by weight of neurosporene relative to the total weight of the composition. Preferably, neurosporene represents 50% or more by weight of the total carotenoids present in the composition.

According to certain particular modes, the neurosporene is used in combination with one or more other carotenoids, preferably with phytoene, phytofluene, lycopene, zeta-carotene and/or gamma-carotene, and more particularly preferably with phytoene, lycopene and zeta-carotene. Preferably, the composition does not comprise methoxyneurosporene, spheroidenone and/or epsilon-carotene, and more particularly preferably does not comprise spheroidenone.

The composition may comprise one or more additional cosmetic active ingredients, preferably chosen from moisturizing agents, antioxidants, anti-ageing agents, tensioning agents, emollients, UV-screening agents, plant extracts, algal extracts, essential oils, disinfecting agents, antibiotics, antifungals or antiparasitics, astringents or else agents for stimulating cell renewal.

The present invention also relates to a cosmetic process for combatting a harmful effect of blue light and/or of UVA radiation, preferably combatting skin ageing or a symptom thereof, preferably the appearance of pigment spots, skin dehydration, the formation of wrinkles or fine lines, slackening of the skin or the lack of radiance of the complexion, and/or for promoting epidermal regeneration or renewal and/or for stimulating wound healing, in particular for improving the esthetic appearance of a scarred or damaged area of the skin, comprising the topical application to the skin or the oral administration of a cosmetic composition according to the invention.

The present invention also relates to a process for preparing a cosmetic composition for combatting the harmful effects of blue light and/or UVA radiation, or for promoting epidermal regeneration or renewal and/or for stimulating wound healing, in particular for improving the aesthetic appearance of a scarred or damaged area of the skin, comprising the mixing of at least one cosmetically acceptable excipient with an effective amount of neurosporene, and optionally with phytoene, lycopene, phytofluene, zeta-carotene and/or gamma-carotene.

Preferably, the harmful effect(s) of blue light and/or UVA radiation are chosen from skin ageing and symptoms thereof, preferably the appearance of pigment spots, skin dehydration, the formation of wrinkles or fine lines, slackening of the skin and/or the loss of radiance of the complexion. Preferably, the harmful effect(s) of blue light and/or UVA radiation are chosen from the appearance of pigment spots and skin dehydration.

Alternatively, the harmful effect(s) of blue light and/or of UVA radiation can be chosen from an impairment of the circadian cycle and an impairment of mitochondrial functions. In particular, the present invention relates to the cosmetic use of a composition comprising neurosporene for limiting or preventing an impairment of the circadian cycle caused by blue light.

For these cosmetic applications, the subject for which the composition is intended is preferably a subject (preferably a mammal, and most particularly preferably a human being) not suffering from a skin pathology or an injury requiring therapeutic treatment.

The present invention also relates to a pharmaceutical composition comprising neurosporene, for use in the treatment of a skin injury, preferably a wound, a burn or an abrasion. It also relates to a cosmetic or pharmaceutical composition comprising an effective amount of neurosporene, preferably between 0.00001% and 20% by weight of neurosporene relative to the total weight of the composition, and at least one cosmetically or pharmaceutically acceptable excipient. The composition can also comprise one or more carotenoids, preferably phytoene, phytofluene, lycopene, zeta-carotene and/or gamma-carotene, more particularly preferably phytoene, lycopene, zeta-carotene, phytofluene and gamma-carotene. Preferably, the composition does not comprise methoxyneurosporene, spheroidenone and/or epsilon-carotene. Preferably, the neurosporene represents 50% or more by weight of the total carotenoids present in the composition.

### FIGURE LEGENDS

FIG. 1: graph representing the production of reactive oxygen species (ROSs), after exposure to UVA radiation, by nontreated (NT) keratinocytes, and keratinocytes treated with α-tocopherol or with increasing concentrations of neurosporene. The results are expressed as percentage relative to the ROSs produced in the nontreated keratinocytes.

FIG. 2: graph representing the ROS production, after exposure to UVA radiation, by nontreated (NT) keratinocytes, and keratinocytes treated with α-tocopherol or with increasing concentrations of bacterial extract comprising neurosporene. The results are expressed as percentage relative to the ROSs produced in the nontreated keratinocytes.

FIG. 3: graph representing the ROS production after exposure to blue light, by nontreated (NT) keratinocytes, and keratinocytes treated with α-tocopherol or with increasing concentrations of neurosporene. The results are expressed as percentage relative to the ROSs produced in the nontreated keratinocytes.

FIG. 4: graph representing the ROS production after exposure to blue light, by nontreated (NT) keratinocytes, and keratinocytes treated with α-tocopherol or with increasing concentrations of bacterial extract comprising neurosporene. The results are expressed as percentage relative to the ROSs produced in the nontreated keratinocytes.

FIG. 5: graph representing the ROS production in mitochondria after exposure to blue light in the nontreated (NT) keratinocytes, and in the keratinocytes treated with α-tocopherol or with increasing concentrations of bacterial extract comprising neurosporene. The results are expressed as percentage relative to the ROSs produced in the nontreated keratinocytes.

FIG. 6: histogram representing the filling of the injury measured by cell confluence. The results are expressed as a function of the wound healing obtained for the nontreated keratinocytes. On the left, wound healing of nontreated keratinocytes; in the middle, keratinocytes treated with EGF (epidermal growth factor), and on the right, keratinocytes treated with neurosporene.

FIG. 7: histogram representing the antioxidant power of the various compounds evaluated by the ABTS⋅⁺ radical cation reduction method. The values obtained are expressed as α-tocopherol equivalent in order to determine whether the compounds tested are more or less active than this reference molecule.

FIG. 8: images of the Fontana-Masson staining of biopsies of human skin explants which are nonirradiated or have been irradiated with blue light, which are nontreated or have been treated with vegetable oil alone or with vegetable oil containing 12.5 ppm of neurosporene and 4 ppm of a mixture of carotenoids comprising phytoene, zeta-carotene, phytofluene, lycopene and gamma-carotene. The images were taken with an epifluorescence microscope (Zeiss, Axio Imager Z1, ApoTome, software Zen2 blue edition). The melanin label is visible in black.

FIG. 9: histogram representing the quantification of the melanin after Fontana-Masson staining of biopsies of human skin explants which are nonirradiated or have been irradiated with blue light, which are nontreated or have been treated with vegetable oil alone or with vegetable oil containing 12.5 ppm of neurosporene and 4 ppm of a mixture of carotenoids comprising phytoene, zeta-carotene, phytofluene, lycopene and gamma-carotene. The intensity of the melanin labeling, determined using the image analysis software, was standardized by the length of the basal lamina. The experiment was carried out in triplicate for each condition (nontreated, treatment with vegetable oil alone, treatment with vegetable oil enriched in neurosporene (12.5 ppm)).

FIG. 10: histogram representing the assaying of intracellular ATP before and after irradiation of human keratinocytes with blue light, with or without pretreatment with a bacterial extract comprising neurosporene. The amount of ATP was standardized by the number of live cells in each condition.

### DETAILED DESCRIPTION OF THE INVENTION

The inventors have shown in the experimental section below that neurosporene is capable of efficiently combating the various damaging effects that blue light and UVA radiation can have on our skin. In fact, neurosporene can not only efficiently limit the oxidative stress generated by UVA radiation or blue light, but is also capable of aiding the preservation of mitochondrial functions and stimulating keratinocyte migration. It thus participates in preventing the appearance of wrinkles and fine lines or in wound healing. Neurosporene also proves to be efficient for limiting or preventing the appearance of pigment spots, dehydration and inflammation of the skin resulting in the formation of wrinkles/fine lines, in slackening of the skin and/or the loss of radiance of the complexion, and impairments of the circadian cycle due to blue light. These properties make it an active ingredient of choice for cosmetic products intended to preserve the skin against the harmful effect(s) of these types of radiation.

Thus, according to a first aspect, a cosmetic composition comprising neurosporene and at least one cosmetically acceptable excipient.

Neurosporene (C₄₀H₅₈, PubChem ID: 5280789), also called 7,8-dihydro-ψ,ψ-carotene, is a carotenoid pigment of formula (I):

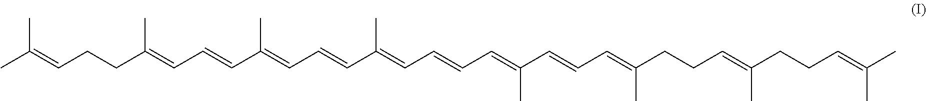 [SMILES: CC(C)=CCC/C(C)=C/C=C/C(C)=C/C=C/C(C)=C/C=C/C=C(C)/C=C/C=C(\C)CC/C=C(\C)CCC=C(C)C]

Neurosporene is an intermediate in lycopene biosynthesis and for various carotenoids.

In general, a cosmetic composition comprises at least one active ingredient which defines the efficacy/the action of the cosmetic product, and at least one excipient that is of use in the formulation of said active ingredient and, most commonly, at least one preservative. In the cosmetic composition according to the invention, the neurosporene is preferably considered to be a cosmetic active ingredient, that is to say that it at least partly defines the action of the product, preferably a preventive action against one or more of the harmful effects of blue light and/or UVA radiation on the skin.

As used herein, the term “harmful effect of blue light and/or of UVA radiation” or “damaging effect of blue light and/or of UVA radiation” refers to a negative effect of blue light and/or of UVA radiation on the subject who is exposed thereto, in particular a negative effect on the skin of the exposed subject. Preferably, the harmful effect induced by blue light and/or UVA radiation is chosen from the group consisting of (i) skin ageing or symptoms thereof preferably chosen from the appearance of pigment spots, skin dehydration, the formation of wrinkles or fine lines, slackening of the skin and/or the loss of radiance of the complexion, (ii) oxidative stress, in particular at the level of the mitochondria and therefore an impairment of mitochondrial functions, and (iii) an impairment of the circadian cycle, and a combination thereof. Preferably, this term refers to a nonpathological impairment of the skin, induced by blue light and/or UVA radiation, that is to say any nonpathological modification of the visual appearance and/or of the mechanical properties of the skin. The nonpathological impairments of the skin can in particular be chosen from skin ageing or one or more symptoms thereof, preferably chosen from the appearance of pigment spots, skin dehydration, the formation of wrinkles or fine lines, slackening of the skin and/or the loss of radiance of the complexion, and more particularly preferably chosen from the appearance of pigment spots and/or skin dehydration.

As used herein, the term “UVA radiation” refers to ultraviolet radiation having a wavelength of from 315 nm to 400 nm, preferably from 350 nm to 390 nm. As used herein, the term “blue light” refers to a radiation having a wavelength of from 401 nm to 500 nm, preferably from 420 nm to 480 nm.

The composition according to the invention preferably comprises an effective amount of neurosporene. This amount is sufficient to prevent or limit one or more harmful effects of blue light and/or UVA radiation, preferably blue light. It can vary according to the applications envisioned. According to certain embodiments, this amount is sufficient to prevent or limit oxidative stress and/or skin dehydration and/or the appearance of pigment spots and/or inflammation, induced by blue light and/or UVA radiation, and/or to stimulate keratinocyte migration, preferably in the area of the body where the cosmetic composition is applied when said composition is applied topically. Preferably, the amount of neurosporene present in the composition is sufficient to prevent or limit the oxidative stress induced by blue light and/or UVA radiation, and to stimulate keratinocyte migration, preferably in the area of the body where the cosmetic composition is applied when said composition is applied topically. The activity on oxidative stress and on keratinocyte migration can be evaluated by any technique known to those skilled in the art, in particular by the methods described in the experimental section. The other activities can be measured by exposing human skin explants to blue light in the presence and in the absence of the compound to be tested in a predetermined amount and by measuring the expression levels of relevant genes. The activity of preventing/limiting the appearance of pigment spots can be evaluated by measuring the expression levels of melanogenesis genes, such as the TYR (NCBI GeneID: 7299), OPN3 (NCBI GeneID: 23596) and/or MLANA (NCBI GeneID: 2315) genes or by direct observation of the amount of melanin produced in the explants. The activity of preventing/limiting skin dehydration can be evaluated by measuring the expression levels of hydration genes such as AQP3 (NCBI GeneID: 360) and/or FLG (NCBI GeneID: 2312). The activity of preventing/limiting inflammation can be evaluated by measuring the expression levels of inflammation genes such as HO1 (NCBI GeneID: 3162), IL6 (NCBI GeneID: 3569) and/or IL1a (NCBI GeneID: 3552).

Preferably, the composition comprises between 0.00001% and 20% by weight of neurosporene relative to the total weight of the composition, and more particularly preferably between 0.001% and 10% by weight of neurosporene relative to the total weight of the composition, and preferably more than anything between 0.001% and 2% by weight of neurosporene relative to the total weight of the composition. According to one particular embodiment, the composition comprises between 0.1% and 1% by weight of neurosporene relative to the total weight of the composition.

The neurosporene used in the composition can be of natural origin, that is to say synthesized by an organism (for example by a plant, a microorganism or an alga), or of synthetic origin, that is to say synthesized by a process involving at least one in vitro chemical modification step. It can be added to the cosmetic composition in a purified form (preferably with a purity greater than 90%, 95% or 99%) or in a nonpurified form, in particular in the form of an extract, such as a plant extract, an algal or microalgal extract, or a microorganism extract, preferably a bacterial extract, said extract being obtained from a plant, alga, microalga or microorganism which synthesizes this compound. The methods for obtaining such extracts are well known to those skilled in the art. Preferably, the extract is a microorganism extract, more particularly preferably a bacterial extract. Such extracts and also the methods for obtaining them are in particular described in patent application FR3049462.

The composition according to the invention can comprise a single carotenoid, namely neurosporene, or a combination of various carotenoids. In particular, it can comprise not only neurosporene, but also phytoene, phytofluene, beta-carotene, lycopene, zeta-carotene and/or gamma-carotene, preferably phytoene, phytofluene, lycopene, zeta-carotene and/or gamma-carotene, and more particularly preferably phytoene, lycopene and/or zeta-carotene. These compounds belong to the lycopene biosynthesis pathway and are often combined in plant or bacterial extracts comprising neurosporene. The relative amounts of these carotenoids can vary according to extracts. According to one preferred embodiment, the cosmetic composition according to the invention comprises neurosporene, phytoene, lycopene, zeta-carotene, phytofluene and gamma-carotene. According to one most particularly preferred embodiment, the cosmetic composition according to the invention comprises neurosporene, phytoene, lycopene and zeta-carotene. Alternatively or additionally, the composition can comprise one or more other carotenoids, such as astaxanthin, zeaxanthin, canthaxanthin, adonirubin, adonixanthin, echinenone, cryptoxanthin, 2-deoxydeinoxanthin or deinoxanthin. Preferably, the composition does not comprise methoxyneurosporene, spheroidenone and/or epsilon-carotene, and most particularly preferably comprises neither methoxyneurosporene nor spheroidenone, and optionally does not comprise epsilon-carotene. Preferably, when the composition comprises several carotenoids, neurosporene is the major carotenoid (present in the largest amount relative to each of the other carotenoids present). More particularly preferably, the neurosporene represents 50% or more by weight of the total carotenoids present in the composition.

The cosmetic composition according to the invention can be in particular a hygiene product (for example deodorant, shower gel, soap, shampoo), or a cosmetic care product (for example antiwrinkle cream, day cream, night cream, moisturizing cream, floral water, scrub, milk, beauty mask, lip balm, tonic), a hair product (for example conditioner, relaxing product, gel, oil, lacquer, mask, dye), a perfume (for example eau de cologne, eau de toilette, perfume), a makeup product (for example concealer, self-tanning agent, eyeliner, face powder, foundation, kohl, mascara, powder, skin-bleaching product, lipstick and/or nail varnish), a sun product (for example aftersun and sun creams, oils or lotions) and/or a nutricosmetic product (cosmetic product intended for oral administration). According to preferred embodiments, the cosmetic composition according to the invention is a product intended for topical application to the skin. Preferably, the cosmetic composition is a hygiene product, a cosmetic care product, a makeup product or a sun product. More particularly preferably, the cosmetic composition is a cosmetic care product, a makeup product or a sun product. The cosmetic composition according to the invention is in general a leave-on composition.

The cosmetic composition according to the invention can comprise one or more additional cosmetic active ingredients, preferably chosen from moisturizing agents, antioxidants, anti-ageing agents, tensioning agents, emollients, UV-screening agents, plant extracts, algal extracts, bacterial extracts, essential oils, disinfecting agents, antibiotics, antifungals or antiparasitics, astringent agents or else cell renewal-stimulating agents. Preferably, the composition comprises at least one additional cosmetic active ingredient which is an anti-ageing agent, a moisturizing agent, antioxidants or a tensioning agent.

According to one particular embodiment, the composition comprises at least one additional cosmetic active ingredient which is an anti-ageing agent, in particular an agent capable of preventing or treating wrinkles, slackening of the skin and/or the appearance of pigment spots. The anti-ageing agent may be for example a free-radical scavenger, an agent for stimulating keratinocyte and/or fibroblast differentiation and/or proliferation; an agent for stimulating collagen synthesis, an agent for preventing collagen degradation, an anti-glycation agent, a depigmenting and/or melanogenesis-inhibiting agent, or a mixture thereof.

Alternatively or additionally, the composition may also comprise at least one additional cosmetic active ingredient which is a tensioning agent, in particular an agent capable of tensioning the skin via mechanical action and of thus reducing the appearance of wrinkles and fine lines. The tensioning agent is in general a polymer, for example a polysaccharide.

The cosmetic composition according to the invention comprises at least one cosmetically acceptable excipient, that is to say a carrier compatible with the skin, the mucous membranes, the nails and/or the hair or compatible with an oral administration when the composition is a nutricosmetic product. The excipient(s) used define the galenical form of the composition. The composition can be provided in any galenical form, preferably in a galenical form used for topical application, such as an aqueous, aqueous-alcoholic or oily solution, an oil-in-water or water-in-oil or multiple emulsion, an aqueous or oily gel, a liquid, pasty or solid anhydrous product, a dispersion of oil in an aqueous phase using spherules, in particular nanospheres, nanocapsules or liquid vesicles. According to certain particular embodiments, the cosmetic composition is a cream, an ointment, a milk, a lotion, a serum, a paste or a foam. It can optionally be applied to the skin in the form of an aerosol. It can also be in solid form, for example in the form of a stick. Alternatively, the composition may be provided in a galenical form suitable for oral administration, in particular in the form of tablets, capsules, gel capsules, granules, suspensions, emulsions or solutions. According to one particular embodiment, the composition comprises at least one excipient which is an oil, preferably a plant oil suitable for topical administration and oral administration.

According to a second aspect, the present invention also relates to a process for preparing a cosmetic composition according to the invention, in particular a cosmetic composition for combatting one or more harmful effects of blue light and/or UVA radiation, and/or for promoting epidermal regeneration or renewal, and/or for stimulating keratinocyte migration and/or for stimulating skin wound healing, in particular for improving the esthetic appearance of a scarred or damaged area of the skin, comprising the mixing of at least one cosmetically acceptable excipient, with an effective amount of neurosporene, and optionally with phytoene, lycopene, phytofluene, zeta-carotene and/or gamma-carotene.

The embodiments described above are also to be envisioned in this aspect.

The neurosporene used for preparing the cosmetic composition may be neurosporene in a purified form (preferably with a purity greater than 90%, 95% or 99%) or in a nonpurified form, in particular in the form of an extract, in particular a plant extract or a microorganism extract, preferably a bacterial extract, obtained from a plant or from a microorganism which synthesizes this compound. Preferably, the neurosporene used for preparing the cosmetic composition is in the form of an extract obtained from a microorganism, preferably a bacterium, which synthesizes neurosporene. The neurosporene concentration in the extract can vary according to the nature of the microorganism or the culture conditions. The strain used may be a wild-type strain (not genetically modified) or a genetically modified strain. The extract may comprise one or more other carotenoids, in particular phytoene, lycopene, phytofluene, zeta-carotene and/or gamma-carotene.

In the preparation process according to the invention, the neurosporene (pure or in a nonpurified form) is mixed with at least one cosmetically acceptable excipient, and optionally with at least one additional cosmetic active ingredient and/or at least one preservative, that is to say a substance which is exclusively or mainly intended to prevent the development of microorganisms in a composition. Preferably, in the preparation process according to the invention, the neurosporene (pure or in a nonpurified form) is mixed with at least one cosmetically acceptable excipient which is an oil, preferably a plant oil suitable for topical administration and oral administration.

By virtue of the action of blue light and UVA radiation on the skin, there are many harmful effects of these radiations. Indeed, as mentioned above, they are capable of causing not only an acceleration of skin ageing which results for example in the appearance of pigment spots, skin dehydration, the formation of wrinkles or fine lines, slackening of the skin and/or the loss of radiance of the complexion, but also an oxidative stress capable of impairing mitochondrial functions and a desynchronization of the circadian cycle. In point of fact, the inventors have shown that neurosporene has the ability to act on these various mechanisms.

Thus, according to another aspect, the present invention relates to the cosmetic use of a composition comprising neurosporene for protecting the skin against one or more of the harmful effects of blue light and/or UVA radiation. It also relates to a composition comprising neurosporene for use for protecting the skin against one or more of the harmful effects of blue light and/or UVA radiation. As used herein, the term “for protecting the skin against one or more of the harmful effects of blue light and/or UVA radiation” refers to an activity aimed at preventing or limiting the effect(s). Alternatively or additionally, the composition may also be used for promoting epidermal regeneration or renewal, and/or stimulating keratinocyte migration and/or for stimulating skin wound healing, in particular for improving the esthetic appearance of a scarred or damaged area of the skin.

The present invention also relates to a cosmetic process for combating one or more harmful effects of blue light and/or UVA radiation, comprising the topical application to the skin of a composition comprising neurosporene. As used herein, the term “for combatting one or more harmful effects of blue light and/or UVA radiation” refers to an activity aimed at preventing or limiting the effect(s). Alternatively or additionally, the process may also be a cosmetic process for promoting epidermal regeneration or renewal, and/or stimulating keratinocyte migration and/or for stimulating skin wound healing, in particular for improving the esthetic appearance of a scarred or damaged area of the skin. The present invention also relates to a cosmetic process for combatting one or more harmful effects of blue light and/or UVA radiation, and/or for promoting epidermal regeneration or renewal, and/or stimulating keratinocyte migration and/or for stimulating skin wound healing, in particular for improving the esthetic appearance of a scarred or damaged area of the skin, comprising the oral administration of a nutricosmetic product comprising neurosporene.

Preferably, the composition used is a cosmetic composition according to the invention and as defined above. The embodiments described above are also to be envisioned in this aspect.

The harmful effects of blue light and/or UVA radiation generally lead to skin ageing. This ageing results in particular in the appearance of pigment spots, skin dehydration, the formation of wrinkles or fine lines, slackening of the skin and/or the loss of radiance of the complexion. Thus, in the context of the cosmetic use or of the cosmetic process according to the invention, the composition is preferably used for preventing or limiting the appearance of pigment spots, skin dehydration, the formation of wrinkles or fine lines, slackening of the skin and/or the loss of radiance of the complexion. More particularly preferably, the composition is used for preventing or limiting the appearance of pigment spots and/or skin dehydration.

When the composition is intended for topical application, said composition can be applied to at least one area of the body which exhibits signs of skin ageing or which is liable to be exposed to blue light and/or to UVA radiation, in particular the face, the neck or the neckline. It can also be applied to the entire body. Preferably, the composition is applied to the body of an adult individual, preferably an individual more than 30, 40, 50 or 60 years old. When the composition is a nutricosmetic composition, it is preferably administered to an adult individual, preferably to an individual more than 30, 40, 50 or 60 years old.

Alternatively or additionally, the composition may also be used for preventing or limiting the impairments of the circadian cycle caused by blue light. Indeed, by virtue of its action on certain receptors such as cytochrome CRY2 (cytochrome circadian regulator 2), blue light plays a major role in regulating the circadian cycle. The exposure to unnatural sources of blue light disrupts this regulation system. In point of fact, the inventors have shown that neurosporene is capable of limiting or preventing these disruptions. Thus, a composition according to the invention applied to the skin or orally administered makes it possible to limit or prevent the impairments of the circadian cycle caused by blue light. There may be many consequences, in particular an improvement in sleep. The activity on the circadian cycle can be measured by exposing human skin explants to blue light in the presence and in the absence of the compound to be tested, in a determined amount, and by measuring the expression levels of genes involved in the regulation of the circadian cycle, such as CRY2 (NCBI GeneID: 1408) and/or BMAL (NCBI GeneID: 406).

The composition may also be used for promoting epidermal regeneration or renewal, and/or stimulating keratinocyte migration and/or for stimulating skin wound healing in applications which can be distinct from the effects of blue light, UVA radiation or skin ageing. Indeed, the composition may be used on a scarred or damaged area of the skin in order to improve the esthetic appearance thereof. In this case, the desired effect is that of improving the esthetic appearance of this area, for example by reducing a scar or by improving the texture of the skin. The skin may have been damaged in many ways, in particular by a wound, preferably a superficial wound, a burn, for example sunburn, or an abrasion, which may be traumatic or in the context of an esthetic treatment. In its cosmetic applications, the composition is not used for treating this damage, but for improving the esthetic consequences thereof.

The composition may be applied one or more times a day, for example in the morning and/or in the evening.

In all the cosmetic applications of the present invention, the subject to whom the composition is administered is preferably a healthy subject, in particular a subject not suffering from a skin pathology or an injury requiring a therapeutic treatment. The cosmetic applications are understood to be nontherapeutic.

The inventors have demonstrated that neurosporene is capable of stimulating keratinocyte migration and of stimulating wound healing.

Thus, according to another aspect, the present invention relates to
a pharmaceutical composition comprising neurosporene for use in the treatment of a skin injury;
the use of neurosporene for the preparation of a medicament for treating a skin injury; and
a method for treating a subject suffering from a skin injury, comprising the administration to said subject of a therapeutically effective amount of neurosporene.

The skin injury is preferably an injury of the epidermis, in particular a wound, preferably a superficial wound, a burn, preferably a 1st-degree or 2nd-degree burn, an abrasion, in particular a superficial traumatic abrasion. Preferably, the skin injury to be treated is a superficial injury which does not extend to the lower layers of the skin, namely to the dermis and to the hypodermis.

The subject to be treated is an animal, preferably a mammal. According to one particular embodiment, the subject to be treated is a human being.

The term “therapeutically effective amount” as used herein refers to the amount of neurosporene required to obtain a stimulation of keratinocyte migration and therefore a wound healing activity. The amount of neurosporene to be administered and also the duration of the treatment are evaluated by those skilled in the art according to the physiological state of the subject to be treated and the injury to be treated, for example according to the seriousness or the area affected. Typically, the pharmaceutical composition comprising neurosporene is administered one or more times a day for at least one week.

The present invention also relates to a pharmaceutical composition comprising neurosporene and at least one pharmaceutically acceptable excipient. The pharmaceutically acceptable excipients that can be used in the composition according to the present invention are well known to those skilled in the art (Remington's Pharmaceutical Sciences, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3^{rd} edition, A. Kibbe, Ed., Pharmaceutical Press[2000]).

The pharmaceutical composition according to the invention can be suitable for oral, sublingual, cutaneous, subcutaneous, intramuscular, intravenous, topical, local, intratracheal, intranasal, transdermal, rectal, vaginal, intra-ocular or intra-auricular administration. The pharmaceutical composition according to the invention may be in the form of tablets, capsules, gel capsules, granules, suspensions, emulsions, solutions, gels, pastes, ointments, creams, plasters, potions, suppositories, enemas, injectable compositions, implants, patches, sprays or aerosols. Preferably, the pharmaceutical composition according to the invention is suitable for topical administration to the skin.

Preferably, the pharmaceutical composition according to the invention comprises between 0.00001% and 20% by weight of neurosporene relative to the total weight of the composition, and particularly preferably between 0.001% and 10% by weight of neurosporene relative to the total weight of the composition, and preferably more than anything between 0.001% and 2% by weight of neurosporene relative to the total weight of the composition. According to one particular embodiment, the composition comprises between 0.1% and 1% by weight of neurosporene relative to the total weight of the composition.

The neurosporene can be added to the pharmaceutical composition in a purified form or in the form of an extract, in particular a plant extract, an algal or microalgal extract, or a bacterial extract, said extract being obtained from a plant, alga, microalga or microorganism, preferably a bacterium, which synthesizes this compound.

The pharmaceutical composition according to the invention can comprise a single carotenoid, namely neurosporene, or a combination of various carotenoids. In particular, it can comprise not only neurosporene, but also phytoene, phytofluene, beta-carotene, lycopene, zeta-carotene and/or gamma-carotene, preferably phytoene, phytofluene, lycopene, zeta-carotene and/or gamma-carotene, and more particularly preferably phytoene, lycopene and/or zeta-carotene. The relative amounts of these carotenoids can vary. According to one preferred embodiment, the pharmaceutical composition according to the invention comprises neurosporene, phytoene, lycopene, phytofluene, zeta-carotene and gamma-carotene, preferably neurosporene, phytoene, lycopene and zeta-carotene. Alternatively or additionally, the composition can comprise one or more other carotenoids, such as astaxanthin, zeaxanthin, canthaxantin, adonirubin, adonixanthin, echinenone, cryptoxanthin, 2-deoxydeinoxanthin or deinoxanthin. Preferably, when the composition comprises several carotenoids, neurosporene is the major carotenoid (present in the largest amount relative to each of the other carotenoids present). More particularly preferably, neurosporene represents 50% or more by weight of the total carotenoids present in the composition.

The pharmaceutical composition according to the invention can also comprise one or more additional active substances, for example antibiotics, antiparasitics, antifungals or antivirals, steroidal or nonsteroidal anti-inflammatories, anesthetic agents, antipruritic agents and/or free-radical scavengers.

All the references cited in this description are incorporated into the present application by way of reference. Other characteristics and advantages of the invention will appear more clearly on reading the following examples given by way of illustration and in a nonlimiting manner.

### Examples

Neurosporene

The neurosporene used in the examples was a purified neurosporene solution (CaroteNature; CAS No. 502-64-7) or a bacterial extract comprising various carotenoids, namely neurosporene, phytoene, lycopene, zeta-carotene, phytofluene and gamma-carotene, neurosporene being the major carotenoid of this extract.

Antioxidant Tests—Detection of ROSs Under Irradiation with UVA Radiation or Blue Light.

HaCaT human keratinocytes were seeded into 96-well microplates in a proportion of 10 000 cells/well in DMEM medium supplemented with 10% of FCS (fetal calf serum) and incubated at 37° C. in 5% CO₂ and 95% humidity for 24 hours. After this period of recovery, the cells were pretreated with either 43 μg/ml of α-tocopherol or with an increasing range of neurosporene (from 0 to 4 μg/ml) or with an increasing range of bacterial extract comprising neurosporene (from 0 to 15 μg/ml) for one hour. At the end of the pretreatment, 5 μM of “CellROX deep red” (a label which becomes fluorescent in the presence of reactive oxygen species (ROS)) and Hoechst (10 μg/ml) (which becomes fluorescent when it is bound to the DNA in the nucleus) were added to the medium. Immediately afterwards, the cells were treated either with UVA radiation (20 J/cm 2), or with a blue light (460 nm, 150 J/cm 2). The cells were then washed three times in 1×DPBS buffer and observed under a fluorescence microscope (CellInsight CX7, ThermoFisher). The fluorescence signals were then analyzed using the HCS Studio software (ThermoFisher) in order to quantify the ROSs produced in the cytosol.

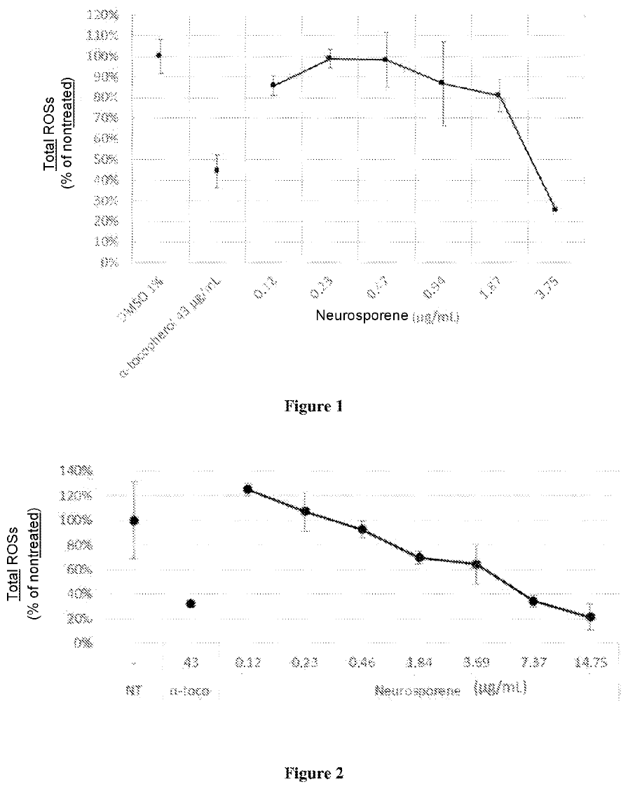

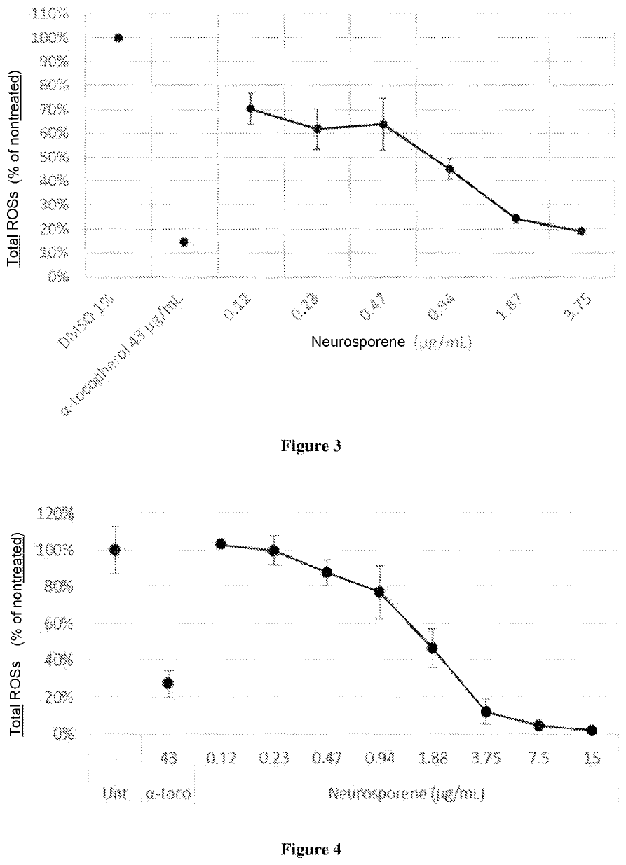

The results of these tests are presented in FIGS. 1 to 4 and show that neurosporene has a strong antioxidant power against blue light and UVA radiation. This activity is much greater than that of α-tocopherol.

Antioxidant Tests—Detection of Mitochondrial ROSs Under Irradiation with Blue Light.

HaCaT human keratinocytes were seeded into 96-well microplates in a proportion of 10 000 cells/well in DMEM medium supplemented with 10% of FCS and incubated at 37° C. in 5% CO₂ and 95% humidity for 24 hours. After a period of recovery, the cells were pretreated with either 43 μg/ml of α-tocopherol or with an increasing range of neurosporene (from 0 to 15 μg/ml) for one hour. At the end of the pretreatment, 200 nM of Mitotracker (a label which targets the mitochondria and becomes fluorescent in the presence of ROSs and more particularly the superoxide anion) and Hoechst (10 μg/ml) were added to the medium.

Immediately afterwards, the cells were treated with blue light (460 nm, 150 J/cm 2). The cells were then washed three times in 1×DPBS buffer and observed under a fluorescence microscope (CellInsight CX7, ThermoFisher). The fluorescence signals were then analyzed using the HCS Studio software (ThermoFisher) in order to quantify the ROS produced in the mitochondria.

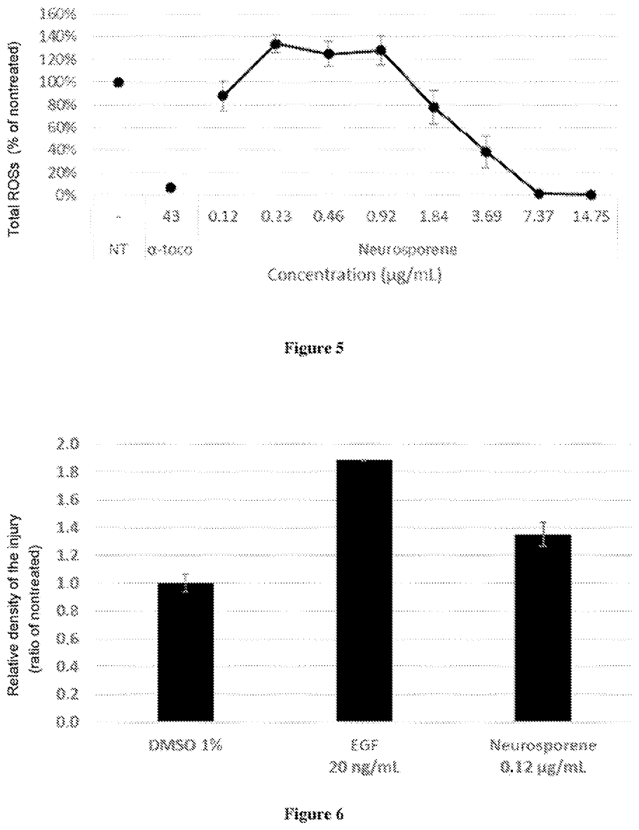

The results of these tests are presented in FIG. 5 and show that neurosporene has a strong antioxidant power against the mitochondrial oxidative stress generated by blue light, with a 98% reduction in the ROSs produced in the presence of 15 μg/ml of bacterial extract containing neurosporene. This activity is much greater than that of α-tocopherol, demonstrating a strong power of neurosporene in the mitochondrial function protection.

Wound Healing Tests

HaCaT human keratinocytes were seeded into 96-well microplates in a proportion of 33 000 cells/well in DMEM medium supplemented with 10% of FCS and incubated at 37° C. in 5% CO2 and 95% humidity for 24 hours. Once confluence was reached, the cells were washed three times in FCS-free DMEM in order to remove from the wells the growth factors present in the complete medium. The keratinocyte monolayers were then scratched using a “WoundMaker” (Essen Biosciences) which makes it possible to injure cell monolayers in 96-well format. The cells were then washed three times with FCS-free DMEM in order to remove the cell debris. After washing, DMEM medium was added to the wells. EGF (Epidermal growth factor, ng/ml) used as positive control for injury filling or neurosporene (0.12 μg/ml) was added to the wells. The wound healing was observed by phase-contrast microscopy using an “Incucyte ZOOM” (Essen Biosciences), which is an automatic microscope maintained in a CO₂ incubator. The images were then processed using the associated software.

The results of these tests are presented in FIG. 6 and show that neurosporene improves wound healing by 35% compared to the nontreated control.

ABTS (2,2′-azino-bis(3-ethylbenzothiazoline-6-sulphonic acid)) Test

The ABTS test is based on the use of colored cationic radicals. In the presence of a molecule having an antioxidant power, the colored cationic radical is reduced and loses its color. Consequently, the antioxidant power of a molecule can be measured by the decoloration of the radical.

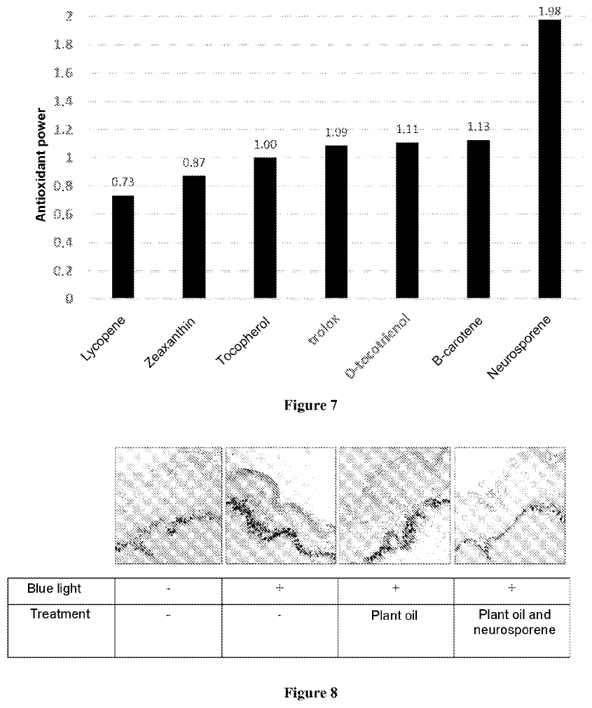

A solution was prepared by mixing ABTS (7 mM), potassium persulfate (2.45 mM) and water (qs 5 ml) and incubated for 16 hours at ambient temperature in the dark. Next, a range of eight concentrations of neurosporene, lycopene, α-tocopherol, 9Z-β carotene, zeaxanthin, trolox and D-tocotrienol was dispensed into 96-well microplates before adding the ABTS solution prepared above. The microplates were then incubated at ambient temperature for 5 min, and the coloration was then analyzed at 250 nm using a microplate reader. The results of this test are presented in FIG. 7 and show that neurosporene has an antioxidant power which is two times greater than that of the reference molecule, α-tocopherol (positive control), and which is significantly greater than all the other carotenoids tested.

Quantification of the Melanin in Human Skin Explants.

Explants of human skin of phototype IV were placed under culture conditions. After 24 h, three conditions were studied: a nontreated control, a topical treatment with refined plant oil alone or a topical treatment with a preparation of refined plant oil containing neurosporene (12.5 ppm of neurosporene and 4 ppm of a mixture of carotenoids, namely phytoene, lycopene, zeta-carotene, phytofluene and gamma-carotene). The products were applied every day (5 mg/cm 2) for 3 days. Each condition was carried out in triplicate.

After 3 days of pretreatment, a blue light stress was applied by irradiation at the wavelength of 447 nm each day for 3 days, corresponding to a dose of 202.6 J/cm². After each irradiation, the explants were again and, depending on the conditions, not treated or treated either with plant oil alone or with the plant oil containing neurosporene.

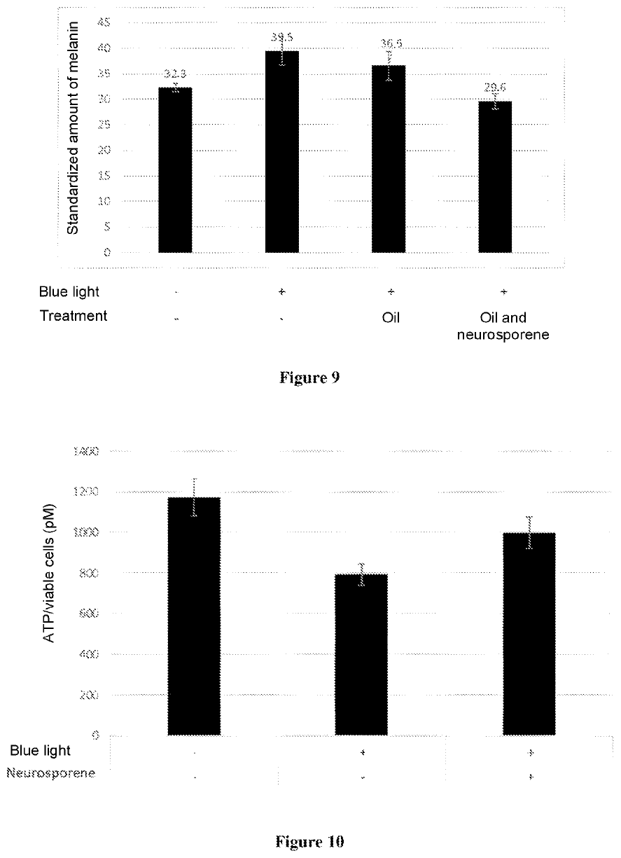

The melanin was then quantified after analysis of images taken with an epifluorescence microscope (Zeiss, Axio Imager Z1, ApoTome, Zen2 software blue edition) and staining according to the Fontana Masson method. The images were then analyzed on ImageJ. The area of melanin staining was standardized by the length of the basal lamina. The experiment was carried out in triplicate for each condition (nontreated, treatment with vegetable oil alone, treatment with vegetable oil enriched in neurosporene). The results of this test are presented in FIGS. 8 and 9 and show (i) that blue light stimulates melanin synthesis (+22% relative to the non-exposed control, p-value<0.05) and (ii) that neurosporene makes it possible to limit the melanin production induced by blue light (−25% compared to the control exposed without treatment, p-value<0.01).

Assaying of ATP after Irradiation with Blue Light

HaCaT human keratinocytes were seeded into 96-well microplates in a proportion of 20 000 cells/well in DMEM supplemented with 10% of fetal calf serum and incubated at 37° C. in 5% CO₂ and 95% humidity for 24 hours. After a period of recovery, the cells were pretreated with a dry bacterial extract taken up in DMSO and containing 0.05 μg/ml of neurosporene and a mixture of carotenoids (phytoene, lycopene, zeta-carotene, phytofluene and gamma-carotene), the neurosporene being the major carotenoid of this extract. The cells were incubated at 37° C. in 5% CO₂ and 95% humidity for 24 hours. Immediately afterwards, the cells were irradiated with blue light (460 nm, 100 J/cm 2). The cells were then washed three times in 1×PBS buffer and permeabilized with 2% triton at 25° C. for 20 min. The supernatants were then collected in order to quantify the ATP by luminescence (Invitrogen™ ATP Determination Kit, Molecular Probes™). The amount of ATP per well is standardized by the amount of live cells. Each condition was carried out in triplicate. The results of this test are presented in FIG. 10 and show that neurosporene makes it possible to limit the ATP reduction induced by blue light.

1-20. (canceled)

21. A composition comprising neurosporene as a cosmetically active ingredient.

22. The composition according to claim 21, wherein the composition is a cosmetic composition or a pharmaceutical composition.

23. The composition according to claim 21, wherein the composition comprises an effective amount of neurosporene.

24. The composition according to claim 21, wherein the composition comprises between 0.00001% and 20% by weight of neurosporene relative to the total weight of the composition.

25. The composition according to claim 21, wherein the composition further comprises one or more other carotenoids.

26. The composition according to claim 21, wherein the composition further comprises:
a) phytoene, lycopene, phytofluene, zeta-carotene and/or gamma-carotene; or
b) phytoene, lycopene and zeta-carotene.

27. The composition according to claim 21, wherein the composition further comprises one or more additional cosmetic active ingredients selected from the group consisting of moisturizing agents, antioxidants, anti-ageing agents, tensioning agents, emollients, UV-screening agents, plant extracts, algal extracts, essential oils, disinfecting agents, antibiotics, antifungals, antiparasitics, astringents, and agents for stimulating cell renewal.

28. The composition according to claim 21, wherein the composition does not comprise methoxyneurosporene, spheroidenone and/or epsilon-carotene.

29. The composition according to claim 21, wherein the neurosporene represents 50% or more by weight of the total carotenoids present in the composition.

30. A cosmetic process for combatting a harmful effect of blue light and/or UVA radiation, and/or for promoting epidermal regeneration or renewal and/or for improving the aesthetic appearance of a scarred or damaged area of the skin, comprising the topical application to the skin or the oral administration of a composition according to claim 21 to a subject in need of treatment.

31. The process according to claim 30, wherein the harmful effect(s) of blue light and/or UVA radiation are selected from skin ageing and symptoms thereof, the appearance of pigment spots, skin dehydration, formation of wrinkles or fine lines, slackening of the skin and/or the loss of radiance of the complexion.

32. The process according to claim 30, wherein the harmful effect(s) of blue light and/or UVA radiation are selected from the appearance of pigment spots and skin dehydration.

33. The process according to claim 30, wherein the subject is not suffering from a skin pathology or an injury requiring a therapeutic treatment.

34. A process for preparing a cosmetic composition for combatting the harmful effects of blue light and/or UVA radiation, or for promoting epidermal regeneration or renewal and/or for improving the aesthetic appearance of a scarred or damaged area of the skin, comprising the mixing of at least one cosmetically acceptable excipient with an effective amount of neurosporene, and optionally with phytoene, lycopene, phytofluene, zeta-carotene and/or gamma-carotene.

35. A method for limiting an impairment of the circadian cycle caused by blue light comprising the topical application to the skin or the oral administration of a composition according to claim 21 to a subject in need of treatment.

36. The method according to claim 35, wherein the subject is not suffering from a skin pathology or an injury requiring a therapeutic treatment.

37. A method of treating a skin injury comprising the topical application of a composition according to claim 21 to a subject having a skin injury.

38. The method according to claim 37, wherein the skin injury is a wound, a burn, or an abrasion.
